(19) United States
(12) Patent Application Publication
(10) Pub. No.: US 2006/0115838 A1
(43) Pub. Date: Jun. 1, 2006

(54) Real-time detection of amplicons using nucleic acid repair enzymes

(75) Inventors: Leonard Bazar, Rockville, MD (US); Jack Chirikjian, Washington, DC (US)

Correspondence Address: FOLEY AND LARDNER LLP;SUITE 500, 3000 K STREET NW, WASHINGTON, DC, 20007, US

(73) Assignee: Trevigen Inc.

(21) Appl. No.: 11/252,986

(22) Filed: Oct. 19, 2005

Related U.S. Application Data
(60) Provisional application No. 60/619,691, filed on Oct. 19, 2004.

(51) Int. Cl. C12Q 1/68; C12P 19/34

(52) U.S. Cl. 435/6; 435/91.2

(57) ABSTRACT

Target nucleic acid sequences can be detected during an amplification reaction in real-time. Detection methods involve conducting an amplification reaction in the presence of at least one nucleic acid repair enzyme and at least one oligonucleotide probe that contains a mismatched or repairable base sequence such that a mismatch occurs at the site of the mismatched or repairable base sequence. Reaction mixtures and kits for detecting target nucleic acids also are provided.

This application claims the priority benefit of U.S. provisional patent application Ser. No. 60/619,691, filed Oct. 19, 2004, which is hereby incorporated by reference.

### BACKGROUND

The ability to rapidly and specifically detect nucleic acids is critically important in both clinical and research applications. In addition, a detection method must detect very low copy numbers of the target molecules. In order to achieve the desired sensitivity and specificity, practitioners employ a variety of amplification techniques.

A common amplification method is the polymerase chain reaction (“PCR”) described in U.S. Pat. No. 4,362,195. PCR allows the amplification of defined DNA sequences from, if necessary, very small quantities of DNA. Amplicons from PCR typically are analyzed using gel electrophoresis (either agarose or acrylamide-based) and a fluorescent, intercalating dye, such as ethidium bromide, to stain for the presence of DNA. This method provides information concerning the number, amount and size of amplicons. Gel electrophoresis, however, requires extensive manual intervention, hazardous reagents and a significant amount of time.

Similarly, one approach conducts an amplification reaction in the presence of a dye that recognizes double-stranded DNA (“dsDNA”), such as Sybrgreen™ available from Molecular Probes, Inc. By measuring an increase in fluorescence, practitioners can monitor the accumulation of amplicons in real-time. The technique permits amplicons to be detected quickly, with fewer cycles, and gives direct information on annealing/melting kinetics. Moreover, since both amplification and detection occur in a single, closed tube, the method reduces the health risk to technicians and the potential contamination of other samples. The technique, however, lacks the ability to confirm that the correct amplicon was amplified as the dye binds to any dsDNA present.

In another approach, labeled detection probes have been incorporated into the amplification reaction. See, e.g., U.S. Pat. No. 5,723,591 (TaqMan Patent). While the technique enables real-time monitoring of amplification reactions, many practitioners find the licensing fee insurmountable and, thus, are precluded from using the approach.

Accordingly, alternative approaches are required for detecting target nucleic acids in real-time.

### SUMMARY

In one embodiment, the invention relates to a method of analyzing for the presence of a target nucleic acid in a sample, the method comprising (A) bringing the sample into contact with a reaction mixture comprised of a plurality of oligonucleotide primers, at least one oligonucleotide probe, and a plurality of nucleic acid enzymes, such that an amplification reaction occurs to produce a plurality of amplicons when the target nucleic acid is present, wherein i) the plurality of oligonucleotide primers is designed for amplifying a region of the target nucleic acid, whereby the amplicons are produced; ii) the probe undergoes hybridization to an amplicon of the plurality of amplicons and contains a mismatched or repairable base sequence such that, in the hybridization, a mismatch occurs at the site of the mismatched or repairable base sequence; and iii) the plurality of nucleic acid enzymes comprises at least a first enzyme having template-dependent nucleic acid polymerase activity and a second enzyme having nucleic acid repair enzyme activity, and then (B) detecting whether the amplicons are present in the reaction mixture.

In other embodiments, the amplification reaction can be, for example, a polymerase chain reaction, a ligase chain reaction, a loop-mediated isothermal amplification, a nucleic acid sequence based amplification, a self-sustained sequence replication, a strand displacement amplification or a transcription mediated amplification. In other embodiments, the second enzyme can be a mismatch repair enzyme, a glycosylase, an apurinic-apyrimidinic (AP) endonuclease or an AP lyase.

The detection methods can be used to simultaneously detect multiple target nucleic acids in a sample.

In another embodiment, the invention relates to a reaction mixture for detecting a target nucleic acid in a sample. In one aspect, the reaction mixture comprises a plurality of oligonucleotide primers, one or more oligonucleotide probes, and a plurality of nucleic acid enzymes, wherein the plurality of oligonucleotide primers are operably designed for amplifying a region of the target nucleic acid, the one or more oligonucleotide probes hybridizes to an amplicon of the target nucleic acid and contains a mismatched or repairable base sequence such that, in the hybridization, a mismatch occurs at the site of the mismatched or repairable base sequence, and the plurality of nucleic acid enzymes comprises at least a first enzyme having template-dependent nucleic acid polymerase activity and a second enzyme having nucleic acid repair enzyme activity. In other aspects, each enzyme of the plurality of enzymes are independently mesophilic or thermophilic. In addition, the second enzyme can be, for example, a mismatch repair enzyme, a glycosylase, an apurinic-apyrimidinic (AP) endonuclease or an AP lyase. The glycosylase can be Uracil DNA glycosylase, E. coli MutY glycosylase, M. thermoautotrophicum TDG or P. aerophilum MIG. In another embodiment, the AP endonuclease is selected from the group consisting of E. coli endonuclease IV, T. maritima endonuclease IV, P. aerophilum endonuclease IV and M. thermoautotrophicum endonuclease IV. Likewise, the plurality of nucleic acid enzymes can comprise a third enzyme such as a mismatch repair enzyme, a glycosylase, an apurinic-apyrimidinic (AP) endonuclease and an AP lyase.

In other embodiments, the reaction mixtures comprise labeled oligonucleotide probes. In one embodiment, the labeled oligonucleotide probe is detectable by fluorescence. In another embodiment, the oligonucleotide probe comprises a first and second label. The first and second labels can be interactive signal generating labels effectively positioned on one or more oligonucleotide probes to quench the generation of detectable signal. The first label can be a fluorophore and the second label can be a quenching agent. In one example, the first label is at the 5′ terminus of the oligonucleotide probe, and the second label is at the 3′ terminus of the oligonucleotide probe. Alternatively, the first and second labels are located internally within the probe on opposite sides of the mismatch site. The kits can comprise multiple oligonucleotide probes possessing distinguishable fluorescent labels and which are directed to different target nucleic acid sequences or different regions within a target nucleic acid.

The reaction mixtures and kits can further comprise the four different deoxyribonucleoside triphosphates and a suitable buffer. In one aspect, the buffer comprises substituents which are cofactors, or which affect pH, ionic strength, etc., of the reaction mixture.

In another embodiment, a kit is provided for detecting a target nucleic acid in a sample. In one aspect, the kit comprises a plurality of oligonucleotide primers, an oligonucleotide probe, and a plurality of nucleic acid enzymes, wherein the plurality of oligonucleotide primers are operably designed for amplifying a region of the target nucleic acid, the one or more oligonucleotide probes hybridize to an amplicon of the target nucleic acid and contains a mismatched or repairable base sequence such that, in the hybridization, a mismatch occurs at the site of the mismatched or repairable base sequence and the plurality of nucleic acid enzymes comprises at least a first enzyme having template-dependent nucleic acid polymerase activity and a second enzyme having nucleic acid repair enzyme activity. Each enzyme of the plurality of enzymes can be independently mesophilic or thermophilic. The second enzyme can be, for example, a mismatch repair enzyme, a glycosylase, an apurinic-apyrimidinic (AP) endonuclease or an AP lyase. In one aspect, the glycosylase can be Uracil DNA glycosylase, E. coli MutY glycosylase, M. thermoautotrophicum TDG or P. aerophilum MIG. In another, the AP endonuclease is selected from the group consisting of E. coli endonuclease IV, T. maritima endonuclease IV and P. aerophilum endonuclease IV. Likewise, the plurality of nucleic acid enzymes can comprise a third enzyme such as a mismatch repair enzyme, a glycosylase, an apurinic-apyrimidinic (AP) endonuclease and an AP lyase.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 shows the hybridization of an oligonucleotide probe to a target nucleic acid to form a mismatch. A nucleic acid repair enzyme then cleaves the probe, which dissociates from the target.

FIG. 2 depicts an oscillating reaction. An excess of oligonucleotide probe hybridizes to available target nucleic acids in the reaction mixture, followed by cleavage at the mismatch site by a nucleic acid repair enzyme. The oligonucleotide probe is designed so that the cleaved probe fragments dissociate from the target nucleic acid. The target then hybridizes to a new intact probe, generating the oscillating reaction. The accumulation of cleaved molecules is linear with the amount of target molecules available.

FIGS. 3A and 3B demonstrate the linking of a nucleic acid repair enzyme to two separate probes designed to hybridize with an amplicon of a target nucleic acid. The nucleic acid repair enzyme comprises two separate individual enzymes, a glycosylase and an AP cleaving enzyme. The glycosylase is linked to an oligonucleotide probe. The AP cleaving enzyme is linked to a second probe which is also complementary to the amplicon and hybridizes at a location adjacent to the oligonucleotide probe. In FIG. 3A, the enzymes are attached to the 5′ ends of the probes, while in FIG. 3B, the enzymes are attached to the 3′ ends.

FIG. 4 depicts an oligonucleotide probe used in combination with second and third probes which hybridize to an amplicon at a location adjacent to the oligonucleotide probe. The glycosylase is attached to the 3′ end of the second probe and the AP cleaving enzyme is attached to the 5′ end of the third probe.

FIG. 5 shows the attachment of glycosylase and an AP cleaving enzyme to the 5′ and 3′ ends of an oligonucleotide probe.

FIG. 7 depicts the structure of natural and artificial AP sites. Structure I depicts a natural AP site; structure II depicts tetrahydrofuranyl (D spacer); structure III depicts propanyl (C3 spacer); structure IV depicts ethanyl; and structure V depicts propanyl.

FIG. 8 shows the detection of LAMP and PCR amplicons using the nucleic acid repair enzyme E. coli endonuclease IV.

FIG. 9 shows the detection of LAMP amplicons in a sample using the nucleic acid repair enzyme E. coli endonuclease IV.

FIG. 10 shows the detection of LAMP amplicons in a sample using the nucleic acid repair enzyme E. coli endonuclease IV.

FIG. 11 shows the real-time detection of HPV-16 DNA in a sample using LAMP amplification, a nucleic acid repair enzyme and an oligonucleotide probe labeled with FRET.

### DETAILED DESCRIPTION

In one aspect, methods of detecting target nucleic acids during an amplification reaction are provided. The technique enables a practitioner to monitor in real-time the accumulation of amplicons in a target-specific manner. In addition, the approach streamlines the detection process and reduces the potential for contaminating test samples.

The methods involve conducting an amplification reaction in the presence of at least one nucleic acid repair enzyme and at least one oligonucleotide probe that contains a mismatched or repairable base sequence such that when hybridizing to a target nucleic acid a mismatch occurs at the site of the mismatched or repairable base sequence. In other embodiments, reaction mixtures and kits for detecting target nucleic acids are provided.

Unless indicated otherwise, all technical and scientific terms are used herein in a manner that conforms to common technical usage. Generally, the nomenclature of this description and the described laboratory procedures, including cell culture, molecular genetics, and nucleic acid chemistry and hybridization, respectively, are well known and commonly employed in the art. Standard techniques are used for recombinant nucleic acid methods, oligonucleotide synthesis, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed, for example, in Sambrook J. and D. W. Russell (2001) MOLECULAR CLONING A LABORATORY MANUAL, 3d ed., Cold Spring Harbor Laboratory Press, Cold Spring Harbor, N.Y.; Ausubel et al. (1989) CURRENT PROTOCOLS IN MOLECULAR BIOLOGY, John Wiley & Sons, Brooklyn, N.Y.; Carl W. Dieffenbach, Gabriela S. Dveksler (2003) PCR PRIMER: A LABORATORY MANUAL, Cold Spring Harbor Laboratory Press, Cold Spring Harbor, N.Y.; and John M. S. Bartlett and David Stirling (2003) PCR PROTOCOLS, Humana Press, Totowa, N.J. Specific scientific methods relevant to the present invention are discussed in more detail below. However, this discussion is provided as an example only, and does not limit the manner in which the methods of the invention can be carried out.

Definitions

The terms “polynucleotide,” and “oligonucleotide” refer to primers, probes, oligomer fragments, oligomer controls and unlabeled blocking oligomers. The terms are generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases.

The terms “target,” “target polynucleotide” or “target nucleic acid” refer to a region of a nucleic acid which is to be amplified, detected, or both.

The terms “amplifying” and “amplification” refer to a process whereby the copy number of a particular nucleic acid is increased. An “amplification reaction” uses purified enzymes to replicate specific nucleic acids. Exemplary amplification reactions include, but are not limited to, a polymerase chain reaction, a ligase chain reaction, a loop-mediated isothermal amplification, a nucleic acid sequence based amplification, a self-sustained sequence replication, a strand displacement amplification, and transcription mediated amplification.

The term “amplicon” refers to a DNA fragment generated by a nucleic acid amplification reaction.

The terms “probe” or “oligonucleotide probe” refer to a short-length of a single-stranded nucleic acid that hybridizes with a target nucleic acid or amplicon of a target nucleic acid and contains one or more detectable labels. In addition, an oligonucleotide probe contains a mismatched or repairable base sequence such that a non-Watson-Crick base pair occurs at the site of the mismatched or repairable base sequence when the probe hybridizes to a target nucleic acid. Preferably, the probe does not contain a sequence complementary to a sequence or sequences used to prime the amplification reaction. Generally the 3′ terminus of the probe is “blocked” to prohibit incorporation of the probe into a primer extension product. Blocking can be achieved by using non-complementary bases or by adding a chemical moiety such as biotin or a phosphate group to the 3′ hydroxyl of the last nucleotide. Such a moiety can, depending upon the selected moiety, serve a dual purpose of also acting as a label for subsequent detection or capture of the nucleic acid attached to the label. Blocking can also be achieved by removing the 3′-OH or by using a nucleotide that lacks a 3′-OH such as a dideoxynucleotide.

As used herein, “primer” refers to an oligonucleotide of a defined sequence. A primer is “operably designed” for amplifying where it is capable of annealing to a target nucleic acid and initiating nucleic acid synthesis. A primer must be sufficiently long to prime the synthesis of extension products in the presence of a nucleic acid enzyme. A primer is preferably single-stranded for maximum efficiency in amplification.

As used herein, “hybridizing” or “hybridization” refers to the widely used technique which exploits the ability of complementary sequences in single-stranded nucleic acid sequences to pair with each other (i.e. anneal) by hydrogen bonding to form a double stranded “hybridization complex.” Hybridization can occur between two complementary or substantially complementary DNA sequences, between single-stranded DNA and complementary or substantially complementary RNA or between two RNA sequences.

The term “nucleic acid enzyme” refers generally to isolated, purified or recombinant enzymes which catalyze a reaction modifying nucleic acids. Exemplary nucleic acid enzymes include enzymes having template-dependent nucleic acid polymerase activity or enzymes having nucleic acid repair activity.

Enzymes having “template-dependent nucleic acid polymerase activity” include those that catalyze the synthesis of nucleic acids using nucleic acid templates, i.e. assembling RNA from ribonucleotides or DNA from deoxyribonucleotides. Known DNA polymerases include, for example, E. coli DNA polymerase I, Thermus thermophilus (Tth) DNA polymerase, Bacillus stearothermophilus DNA polymerase, Thermococcus litoralis DNA polymerase, and Thermus aquaticus (Taq) DNA polymerase. The reaction conditions for catalyzing DNA synthesis with these enzymes are well known in the art.

Enzymes having “nucleic acid repair activity” catalyze the cleavage, at a point of mismatch or needed repair, one strand of a duplex formed by oligonucleotide probe and target polynucleotide.

The terms “multiplex amplification” or “multiplex detection” refer to the amplification and/or detection of two or more target sequences in the same reaction.

The term “label,” as used herein, refers to any atom or molecule which can be used to provide a detectable, and preferably quantifiable, signal. A label can be attached to a nucleic acid. Labels can provide signals detectable by fluorescence, radioactivity, colorimetry, gravimetry, X-ray diffraction or absorption, magnetism, enzymatic activity, and the like.

Discussion

In one embodiment, a method of detecting a target nucleic acid in a sample comprises conducting an amplification reaction with the sample using a reaction mixture comprising a plurality of oligonucleotide primers, one or more oligonucleotide probes, and a plurality of nucleic acid enzymes, wherein the plurality of oligonucleotide primers are operably designed for amplifying a region of the target nucleic acid.

Amplification of Target Nucleic Acids

A variety of techniques can be utilized to amplify target nucleic acids. These amplification reactions typically use isolated, purified or recombinant nucleic acid enzymes to replicate specific nucleic acids. Depending on the amplification reaction, nucleic acid enzymes can have template-dependent nucleic acid polymerase activity, RNA polymerase activity, DNA polymerase activity or reverse transcriptase activity. Both mesophilic and thermophilic enzymes can be used. Exemplary amplification reactions include, but are not limited to, a polymerase chain reaction, a ligase chain reaction, a loop-mediated isothermal amplification, a nucleic acid sequence based amplification, a self-sustained sequence replication, a strand displacement amplification, and a transcription mediated amplification.

In one embodiment, a target nucleic acid can be amplified by a polymerase chain reaction (“PCR”). PCR is a well known amplification reaction for amplifying specific nucleic acid segments. PCR amplifies specific DNA segments by cycles of template denaturation; primer addition; primer annealing and replication using thermostable DNA polymerase. Exemplary protocols for PCR can be found, for example, in U.S. Pat. Nos. 4,683,195 and 4,683,202. A thermostable nucleic acid polymerase is relatively stable to heat when compared, for example, to nucleotide polymerases from E. coli which catalyze the polymerization of nucleoside triphosphates. Generally, the enzyme initiates synthesis at the 3′-end of the primer annealed to the target nucleic acid, and will proceed in the 5′-direction along the target nucleic acid, and if possessing a 5′-to-3′ nuclease activity, hydrolyzing intervening, annealed probe to release both labeled and unlabeled probe fragments, until synthesis terminates. A representative thermostable enzyme isolated from Thermus aquaticus (Taq) is described in U.S. Pat. No. 4,889,818 and a method for using it in conventional PCR is described in Saiki et al., 1988, Science 239:487. Taq DNA polymerase has a DNA synthesis-dependent, strand replacement 5′-3′ exonuclease activity (see Gelfand, “Taq DNA polymerase” in PCR Technology: Principles and Applications for DNA Amplification, Erlich, Ed., Stockton Press, N.Y. (1989)). PCR can be coupled with the use of a nucleic acid enzyme having reverse transcriptase activity to amplify RNA.

In other embodiments, the target nucleic acid can be amplified by ligase chain reaction (“LCR”). LCR is an nucleic acid amplification reaction similar to PCR. LCR differs from PCR as the oligonucleotide probe is the template of the amplicon as opposed to the target nucleic acid. In LCR, two oligonucleotide probes are used per each strand of nucleic acid. The probes are designed to exactly match two adjacent sequences of a specific target DNA. LCR uses both a DNA polymerase enzyme and a DNA ligase enzyme to drive the reaction. Like PCR, LCR requires a thermal cycler to drive the reaction and each cycle results in a doubling of the target nucleic acid molecule. The chain reaction is repeated in three steps in the presence of excess probe: (1) heat denaturation of double-stranded DNA, (2) annealing of probes to target DNA, and (3) joining of the probes by thermostable DNA ligase. Exemplary protocols for LCR are found, for example, in Landegren et al., Science 241:1077-1080 (1988); D. Y. Wu and R. B. Wallace, Genomics 4:560-569 (1989); and F. Barany, PCR Methods Appl. 1:5-16 (1991). LCR can be coupled with the use of a nucleic acid enzyme having reverse transcriptase activity to amplify RNA.

In another embodiment, the target nucleic acid can be amplified by loop-mediated isothermal amplification (“LAMP”). LAMP is an amplification reaction technique with high specificity, efficiency and rapidity under isothermal conditions. In LAMP, a DNA polymerase and four specially designed primers recognize a total of six distinct sequences on a target nucleic acid. An inner primer containing sequences of the sense and antisense strands of the target nucleic acid initiates LAMP. Strand displacement by nucleic acid synthesis primed by an outer primer releases a single-stranded nucleic acid. This single-stand nucleic acid acts as template for nucleic acid synthesis primed by a second set of inner and outer primers. These primers hybridize to the other end of the target, thereby producing a stem-loop DNA structure. In subsequent LAMP cycling, one inner primer hybridizes to the loop on the product and initiates displacement DNA synthesis, yielding the original stem-loop DNA and a new stem-loop DNA with a stem twice as long. Exemplary protocols for LAMP amplification reactions are found, for instance, in Nagamin et al., Clin. Chem. 47(9):1742-1743 (2001); Notomi et al., Nucleic Acids Res. 28(12):E63 (2000), both of which are hereby incorporated by reference. Because it is an isothermal reaction, mesophilic enzymes having nucleic acid polymerase activity can be used to drive the amplification reaction. For example, the DNA polymerase large fragment (Klenow fragment) from E. coli is suitable for use in LAMP.

In another embodiment, the target nucleic acid can be amplified by nucleic acid sequence based amplification (“NASBA”). NASBA is a primer-dependent amplification reaction technique used for the isothermic amplification of nucleic acids. NASBA amplification can be performed on both RNA and DNA target nucleic acids. For RNA, NASBA is initiated by the annealing of an oligonucleotide primer to the RNA target nucleic acid. The 3′ end of the primer is designed such that it is complementary to the target nucleic acid and, at the 5′ end, encodes the T7 RNA polymerase promoter. After annealing, the reverse transcriptase activity of AMV-RT is engaged and a cDNA copy of the RNA target is produced. The RNA portion of the resulting hybrid molecule is hydrolyzed through the action of RNase H. Once sufficiently complete, a second primer, which is complementary to an upstream portion of the RNA target nucleic acid, anneals to the cDNA strand. The DNA-dependent DNA polymerase activity of AMV-RT is engaged again, thereby producing a double stranded cDNA copy of the original RNA target nucleic acid with a fully functional T7 RNA polymerase promoter at one end. NASBA next utilizes T7 RNA polymerase to produce a large amount of anti-sense, single stranded RNA transcripts corresponding to the original RNA target. These anti-sense RNA transcripts can serve as templates for the amplification process, however the primers anneal in the reverse order. For DNA, the process is the same except that an initial heat denaturing step is required before the addition of the enzymes to the reaction mix. An exemplary protocol for NASBA is found in J. Compton, Nature 350:91-92 (1991).

In yet another embodiment, the target nucleic acid can be amplified by self-sustained sequence replication (“3SR”). 3SR is an isothermal amplification reaction which utilizes three enzymatic activities essential to retroviral replication: reverse transcriptase, RNase H, and a DNA-dependent RNA polymerase. Generally, 3SR mimics the retrbviral strategy of RNA replication by means of cDNA intermediates. As such, 3SR accumulates cDNA and RNA copies of the original target nucleic acid. In 3SR, the amplicon accumulates exponentially with respect to time, indicating that newly synthesized cDNAs and RNAs function as templates for a continuous series of transcription and reverse transcription reactions. An exemplary protocol for 3SR, including exemplary enzymes, is found in Guatelli et al., Proc. Natl. Acad. Sci. U.S.A. 87(5):1874-1878 (1990).

In another embodiment, the target nucleic acid can be amplified by strand displacement amplification (“SDA”). SDA is an isothermal amplification reaction technique based upon the ability of a restriction enzyme to nick the unmodified strand of a hem-modified DNA recognition site and the ability of a 5′-3′ exonuclease deficient DNA polymerase to extend the 3′ end at the nick and displace the downstream strand. SDA achieves exponential target nucleic acid amplification by coupling sense and antisense reactions in which strands displaced in a sense reaction serve as target nucleic acids for an antisense reaction and vice versa. Exemplary protocols for SDA are found, for example, in Walker et al., Nucleic Acids Res., 20:1691-1696 (1992); and Walker et al., Proc. Natl. Acad. Sci. U.S.A. 89:392-396 (1992).

The target nucleic acid can also be amplified by transcription mediated amplification (“TMA”). TMA is an amplification reaction technique which utilizes a nucleic acid enzyme having RNA transcription activity and a second nucleic acid enzyme having DNA synthesis activity (i.e. reverse transcriptase) to produce an RNA amplicon from a target nucleic acid. TMA can be used to target both RNA and DNA. An exemplary TMA protocol is found, for example, in Pasternack et al., J. Clin. Microbiol. 35(3):676-678 (1997).

Those skilled in the art will recognize that other amplification reaction methodologies and techniques can be used.

Detection Of Amplicons Using Nucleic Repair Enzymes

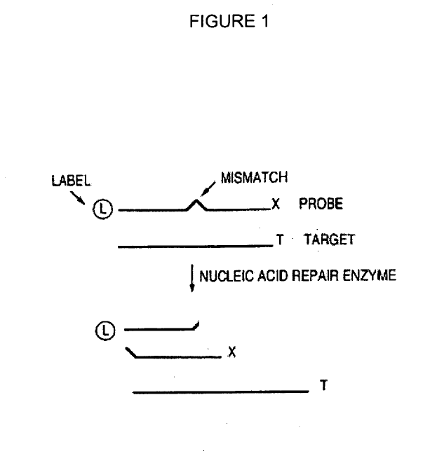

In one embodiment, detection methods involve conducting an amplification reaction in the presence of at least one nucleic acid repair enzyme and one or more oligonucleotide probes that contain a mismatched or repairable base sequence (i.e. a “mutated” sequence) such that when hybridizing to a target nucleic acid a mismatch occurs at the site of the mismatched or repairable base sequence. The nucleic acid repair enzyme recognizes the mismatched or repairable base sequence and cleaves the oligonucleotide probe thereby forming oligonucleotide fragments. As shown in FIG. 1, the fragments spontaneously dissociate from the target nucleic acid at a predetermined temperature.

One embodiment entails hybridizing a single-stranded oligonucleotide probe to a target nucleic acid to form a hybrid, double-stranded polynucleotide. Hybridization occurs under conditions that are “stringent,” i.e., conditions that include a 50-100 mM salt solution at a temperature of (3N-20° C.), where N is the number of nucleotides in the oligonucleotide probe.

Preferably, an oligonucleotide probe is designed not to have self complementary or palindromic regions. Probes also must possess specificity for the target nucleic acid. The parameters for probe design can be found, for example, in Lowe et al., Nucl. Acids Res. 18:1757-1761 (1990); Rychlik et al., loc. cit. 17:8543-8551 (1989); and Rychlik et al., loc. cit. 18:6409-6412 (1990), which discusses probe design as applied to PCR reactions. Probes are designed to be complementary to a target nucleic acid except for a mismatch between the probe sequence and the target nucleic acid sequence. The mismatch should be located internally within the probe and create a non-Watson-Crick base pair when hybridized to a target nucleic acid. The site of the mismatch is selected such that the oligonucleotide probe has a higher melting point than the temperature of the reaction, and the cleaved products have melting points that are lower than the reaction temperature. In another aspect, probes can possess multiple mismatched or repairable base sequences, and thereby possess multiple sites of mismatch on the hybrid, double-stranded polynucleotide.

In one embodiment, the probe is cleaved with a nucleic acid repair enzyme that cleaves, at a point of mismatch, one strand of a duplex formed by oligonucleotide probe and target polynucleotide. Examples of nucleic acid repair enzymes are mutY (Wu et al., Proc. Nat7 Acad. Sci. USA 89:8779-83 (1992)), T/G mismatch-specific nicking enzyme from HeLa nuclear extracts (Wiebauer & Jiricny, Nature 339:234-36 (1989); Wiebauer & Jiricny, loc. cit. 87:5842-45 (1990)), T/G mismatch-specific nicking enzyme from E. coli (Hennecke et al., Nature 353:776-78 (1991)), human yeast all-type enzymes (Yeh et al., J. Biol. Chem. 2667:6480-84 (1991); Chiang & Lu, Nuc. Acids Res. 19:4761-4766 (1981)), Deoxyinosine 3′-endonuclease from E. coli (Yao et al., J. Biol. Chem. 270:28609-16 (1995); Yao et al., J. Biol. Chem. 269:31390-96 (1994)).

In another embodiment, the nucleic acid repair enzyme comprises a glycosylase naturally or recombinantly combined with an AP cleaving enzyme, such as endonuclease or lyase. Such an enzyme can cleave the oligonucleotide probe/target polynucleotide duplex at a point of mismatch. A glycosylase creates a basic sugar (an AP site) at the point of mismatch, which then is cleaved by an AP cleaving enzyme, such as endonuclease or lyase. Illustrative enzymes include, but are not limited to, glycosylases: tag-1, alkA, ung, fpy, mutY, nth, xthA, nfo, recj, uvtA, uvrD, mfd, mutH, mutL, muts, uracil DNA glycosylase, hydroxymethyluracil glycosylase, 5-mC DNA glycosylase, hypoxanthine DNA glycosylase, thymine mismatch DNA glycosylase, 3-mA DNA glycosylase, hydrated thymine DNA glycosylase (endonuclease III), pyrimidine dimer glycosylase.

Such exemplary enzymes can be obtained from a variety of sources. For example, Friedberg et al., DNA REPAIR AND MUTAGENESIS (ASM Press 1995), teaches that uracil DNA glycosylases can be obtained from herpes simplex virus types 1 and 2, equine herpes virus, Varicella zoster virus, Epstein Barr virus, human cytomegalovirus, Mycoplasma lactucae, E. Coli, B. subtilis, M. luteus, B. steorophermaophilus, Thermothrix thirpara, S. pneumoniae, Dictyostelium discoideium, Artenia salina, S. cerevisae, Hordeum vulgare, Zea mays, Triticum vulgare, rat liver mitochondria, calf thymus, human placenta, HeLa S3 cells, and acute leukemia blast cells.

Exemplary AP cleaving enzymes include, but are not limited to, E. coli exonuclease II, E. coli endonuclease IV, Saccharomyes AP endonuclease, Drosphila melanogaster AP endonuclease I and II, human AP endonuclease, human AP lyase, BAP endonuclease, APEX endonuclease, HAP1 and AP endonuclease. Thermophilic AP cleaving enzymes also can be utilized. Examples include, but are not limited to, T. maritima endonuclease IV, P. aerophilum endonuclease IV and M. thermoautotrophicum endonuclease IV.

In another embodiment, cleavage can be effected by using a glycosylase enzyme, as described above, in combination with basic conditions and increased temperature. In this embodiment, increasing pH and temperature effectuates cleavage at the AP site created by the glycosylase enzyme. Suitable parameters for cleavage of the AP site are pH levels of approximately 8 to 14, and temperatures ranging from approximately 50 to 95° C. In addition, these AP endonucleases cleave oligonucleotides containing artificial AP sites, such as those depicted in FIG. 6.

In one embodiment, the detection methods employ a nucleic acid repair enzyme that is thermally stable (i.e., thermophilic), in the sense that the enzyme can function at an elevated temperature, such as from 50 to 80° C. Preferably, the thermally stable nucleic acid repair enzyme can withstand temperatures up to 100° C. for short periods of time.

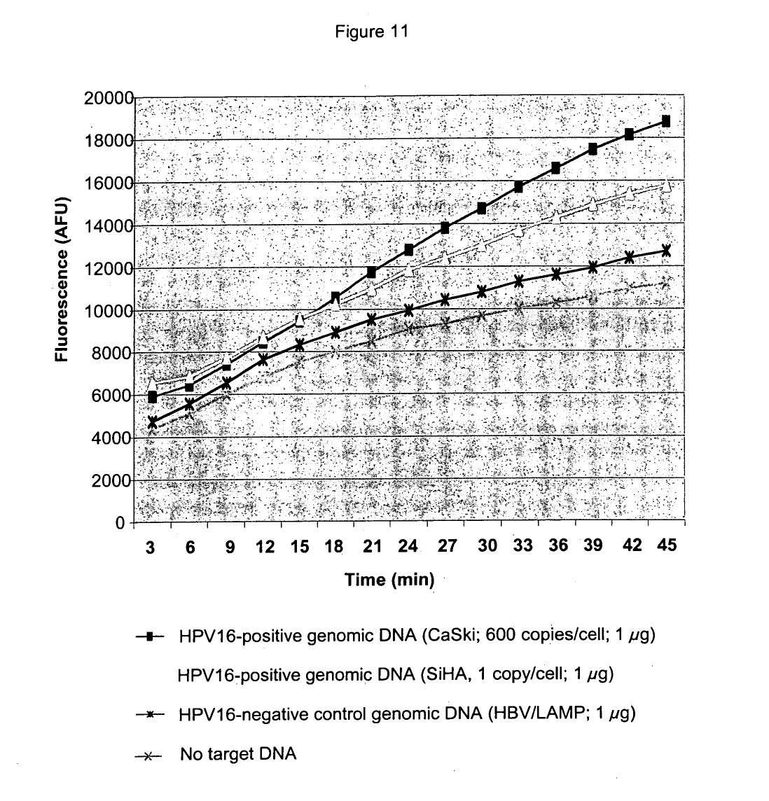

In one embodiment, the detection methods use a thermally stable glycosylase. An example of a thermally stable glycosylase is the ORF10 protein encoded by the DNA sequence of FIG. 11 of U.S. Pat. No. 5,763,178. ORF10 enzyme possesses both base cleaving properties and AP endonuclease activities. Moreover, the AP endonuclease activity of ORF10 can be enhanced, for example, by the technique taught in U.S. Pat. No. 5,763,178.

In addition, ORF10 glycosylase is a homologue of the endonuclease III family. As such, the skilled artisan can identify and isolate genes of the endonuclease III family from other thermophilic bacteria.

In another embodiment, the detection methods utilize a combination of nucleic acid repair enzymes. For example, a nucleic acid repair enzyme can be used in combination with a AP cleaving enzyme. More specifically, mutY can be used in combination with AP cleaving enzymes, such as DNA lyase or DNA AP endonuclease. A combination of enzymes can enhance the speed at which cleavage occurs.

In one embodiment, the nucleic acid repair enzyme is attached to a probe or a combination of probes using techniques known in the art. The attachment of the enzyme to a probe enhances the speed at which cleavage occurs by keeping the nucleic acid repair enzyme in proximity with the probe.

In another embodiment, the nucleic acid repair enzyme is a single enzyme having both glycosylase activity and AP cleaving activity. The enzyme can be attached to a single probe which hybridizes to a target polynucleotide at the point of mismatch. This enzyme can be attached either to the 5′ or to the 3′ end of the probe. The enzyme can be attached to the probe, for example, by linking methods described by Corey et al., Science 238:1401 (1987), and Corey et al., J. Am. Chem. Soc. 111:8523 (1989).

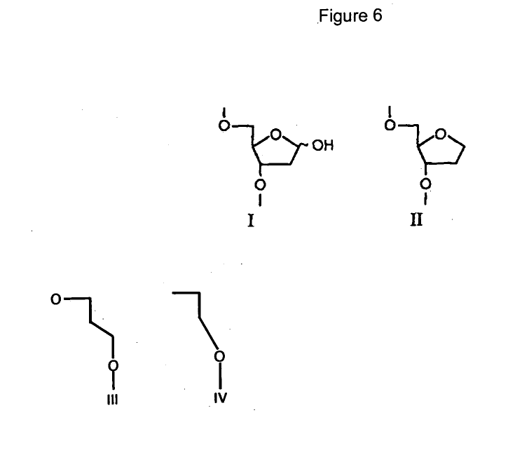

In another embodiment, the detection methods utilize a first and second nucleic acid repair enzyme. The first nucleic acid repair enzyme can possess glycosylase activity, while the second nucleic acid repair enzyme can possess AP cleaving activity. As shown in FIG. 5, the first enzyme can be attached to one end of the probe, and the second enzyme can be attached to the opposite end.

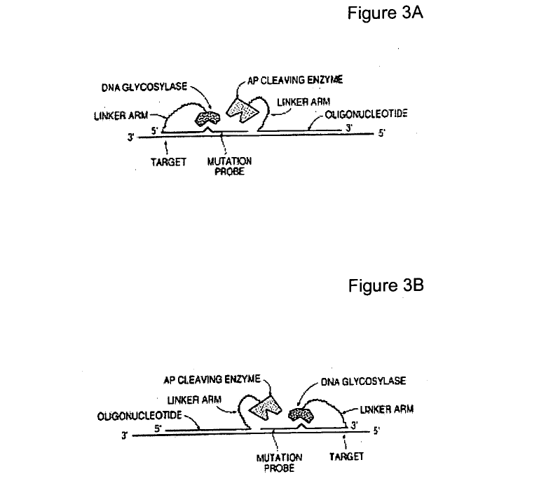

In another embodiment, the detection methods employ two oligonucleotide probes (see FIGS. 3A and 3B). In one example, a first probe is complementary to a target nucleic acid except for a mismatch between probe sequence and the target nucleic acid sequence. A second probe hybridizes to the target polynucleotide at a location adjacent to the first probe, i.e., at a location such that the second probe is close enough to the first probe so that an enzyme attached to the former can effect cleavage of the latter. Preferably, the second probe hybridizes contiguously with the first probe. Alternatively, the second probe can hybridize between one and five base pairs from the first probe.

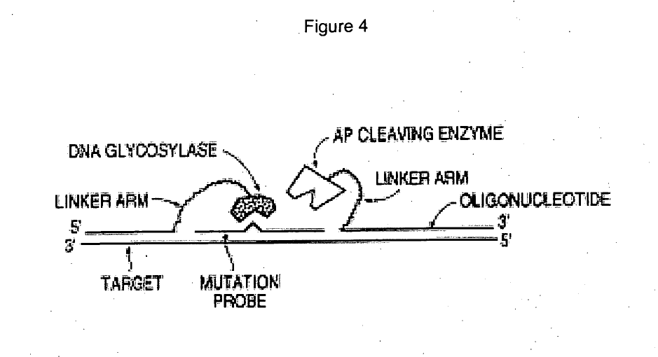

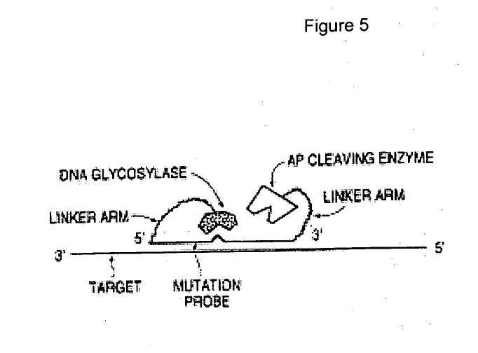

In another embodiment, three probes are used (see FIG. 4). The first probe corresponds to the first probe described above, except that it is not attached to a glycosylase enzyme. Rather, the glycosylase and AP cleaving enzymes are attached to the second and third probes. These second and third probes are designed to hybridize to the target polynucleotide at a location adjacent to the first probe, as described above. Preferably, the second and third probe will hybridize contiguously with the first probe, but on opposite ends. The second and third probes, however, can hybridize between 0 and 5 base pairs from the first probe.

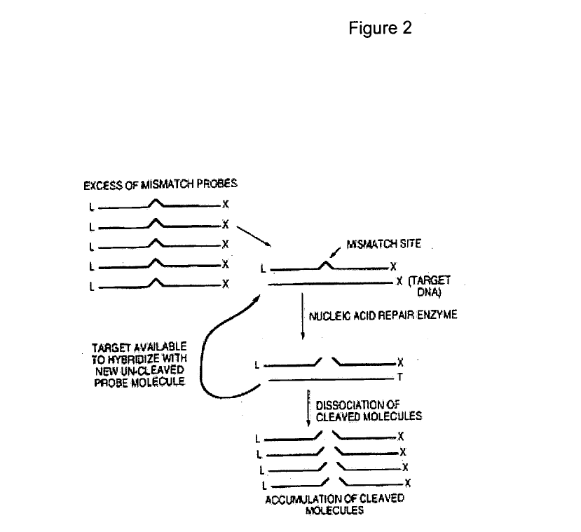

In one embodiment, an oscillation reaction is created whereby the nucleic acid repair enzyme cleaves the oligonucleotide probe, and the shortened, cleaved oligonucleotide fragments dissociate from the target polynucleotide at a predetermined temperature. That is, the oligonucleotide probe is designed so that, at the predetermined temperature, the oligonucleotide fragments dissociate from the target polynucleotide after cleavage by nucleic acid repair enzyme. A cycle or oscillation reaction then occurs because the target polynucleotide hybridizes to another oligonucleotide probe, and the cleavage process is repeated. A schematic of an oscillation reaction is depicted in FIG. 2.

As a consequence, a small number of target polynucleotides can be detected in a sample, since a single target polynucleotide catalyses the formation of a large number of oligonucleotide probe cleavage fragments. The oscillation reaction enables the detection of as little as one molecule of target polynucleotide in a sample. The oscillation reaction can detect from 10-100 target polynucleotide molecules in a sample. Theoretically, the oscillation reaction may detect as little as one target polynucleotide molecule in a sample.

To accommodate the oscillation reaction, a high concentration of oligonucleotide probe is utilized. In this regard, a suitable radiolabeled probe concentration is from 0.01 to 10 pmol. Other concentrations can be used depending on the desired length of autoradiograph exposure times.

Preferably, the oscillating reaction is performed at an isothermal temperature of 3N-20° C., where N is the length of the probe in base pairs. Within this working range the optimal temperature is determined empirically. Preferably, the reaction is performed with 0.01 to 10 pmol of labeled probe, in the presence of either synthetic target sequence or DNA purified from a sample source. This target DNA ranges from 1 to 10¹² molecules.

To reduce the double stranded nature of the target DNA the DNA can be partially degraded with DNAse I to form shorter DNA fragments. The reaction also can be performed in the presence of 10 to 100 pmol of a helix destabilizing molecule in the presence of 5 to 10 mM Mg₂. With the helix destabilizing molecule the operating temperature will need to be empirically determined.

A typical oscillation reaction is performed in a buffer composed of 20 mM Tris-HCl, pH 7.6, 80 mM NaCl, 1 mM dithioerythritol, 1 mM EDTA, pH 8.0, with 5 to 50 units of a nucleic acid repair enzyme. The reaction is allowed to proceed for 20 to 60 minutes. 10 mM EDTA, pH 8.0, 0.025% xylene Cyanol FF, 0.025% Bromophenol blue can be added to stop the reaction.

A typical real-time amplification and detection reaction is performed in a suitable buffer, readily prepared by those of ordinary skill in the art. As one example, a reaction buffer can comprise 20 mM Tris-HCl, pH 7.6, 80 mM NaCl, 1 mM dithioerythritol and 2 mM MgCl₂.

A suitable buffer for detecting amplicons in a LAMP reaction comprises 20 mM Tris-HCl, pH 8.8, 10 mM KCl, 10 mM ammonium sulfate, 10 mM Magnesium sulfate, and 0.1% Triton-X. Each reaction mixture also comprises a plurality of primers for the amplification of target nucleic acids, and about 8 units of Bst DNA polymerase in a 25 μl reaction volume. To 15 μl of the LAMP reaction, 10 units of E. coli Endonuclease IV, and 20 pmoles of a labeled oligonucleotide probe, containing an internal propanyl AP site, are added in a volume of 80 μl in 96-well plates. Fluorescence is read, as the reaction proceeds, as a function of time. The methods also can employ helix destabilizing molecules as described above.

Other exemplary protocols utilizing nucleic acid repair enzymes are found, for instance, in U.S. Pat. Nos. 5,656,430, 5,763,178, and 6,548,247.

As noted above, the detection methods, in one aspect, in involve conducting an amplification reaction in the presence of at least one nucleic acid repair enzyme and at least one oligonucleotide probe that contains a mismatched or repairable base sequence such that when hybridizing to a target nucleic acid a mismatch occurs at the site of the mismatched or repairable base sequence. Typically, the reaction is comprises 0.01 to 10 pmol of one or more oligonucleotide probes.

In one embodiment, fluorescence resonance energy transfer (“FRET”) is used to detect the oligonucleotide probe fragments. FRET is a distance-dependent interaction between the electronic excited states of two dye molecules in which excitation is transferred from a donor molecule to an acceptor molecule without emission of a photon. In one embodiment, the two dye molecules are placed at either end of the labeled oligonucleotide probe. In another example, the two dye molecules are placed internally within the oligonucleotide probe on opposite sides of the mismatched or repairable base sequence. In one embodiment, the donor and acceptor dye molecules are different. In this embodiment, FRET is detected by the appearance of sensitized fluorescence of the acceptor or by quenching of donor fluorescence. In another embodiment, donor and acceptor dye molecules are the same and FRET is detected by the resulting fluorescence depolarization. In addition, multiple oligonucleotide probes directed to different target nucleic acids or regions within a single nucleic acid and having separately detectable labels can be utilized in a single reaction. Exemplary donor and acceptor dye molecule pairs include, but are not limited to those listed in Table 1.

TABLE 1
Exemplary FRET Donor And Acceptor Dye Molecule Pairs

| Donor | Acceptor |
|---|---|
| Fluorescein | Tetramethylrhodamine |
| IAEDANS | Fluorescein |
| EDANS | Dabcyl |
| Fluorescein | Blackhole quencher |
| Fluorescein | QSY 7 and QSY 9 dyes |
| Fluorescein | Iowa Black |

Alternatively, cleaved oligonucleotide probe fragments can be detected by gel electrophoresis. Radiolabeling, fluorescent labeling or other labeling of the oligonucleotides can be used to visualize the oligonucleotide probe fragments. The processed samples are then run on an electrophoresis gel, for example, a 20% polyacrylamide/7M urea-1×TBE gel.

The gel can be analyzed by autoradiograph. The autoradiograph can be scanned electronically, along with control lanes containing different amounts of radiolabeled material. The density of the uncleaved and cleaved oligonucleotide can then be interpolated from electronically scanned data and controls thereby quantifying the amount of cleavage. A similar process can be used for florescence using a fluorimeter. Likewise, chemiluminescence can be detected by autoradiography.

In another embodiment, oligonucleotide probe fragments can be detected using capillary electrophoresis. In this embodiment, the processed samples are separated on a gel in a manner allowing for the quantitation of the amount of cleaved oligonucleotide probe and size determination. Capillary electrophoresis is described, for example, in Guttman et al., J. Chromatography 593:297-303 (1992).

The detection methods can utilize a wide range of labeled oligonucleotide probes. Labels can provide signals detectable by fluorescence, radioactivity, colorimetry, gravimetry, X-ray diffraction or absorption, magnetism, enzymatic activity, and the like. Exemplary labels include, but are not limited to, a fluorophore, a dinitrophenyl group (DNP) or biotin attached on the nucleic acid base. DNP and biotin, as well as some fluorophores, permit for signal amplification through secondary detection techniques, such as the use of biotin-steptavidin amplification, anti-label antibody amplification, tyramide signal amplification, and enzyme-labeled fluorescence amplification.

If the nucleotides used in this embodiment are labeled with a radionucleotide or with some other marker, such as a fluorescent label, then the amount of cleavage can be assayed, using these labeled nucleotides as the signal.

In one embodiment, the 5′ region of the oligonucleotide probe is so designed that it dissociates from the target polynucleotide when the mismatch is cleaved, as shown in FIG. 1.

Single or multiple labels also can be added chemically to oligonucleotides or peptide-nucleic acid conjugates that have been modified with an amine or thiol group during synthesis. Amine-modified oligonucleotide probes can be labeled simply and reliably by amine-reactive succinimidyl esters. The reaction results in a stable amide bond that links the label to the oligonucleotide probe.

In another embodiment, a method of analyzing for the presence of a target nucleic acid in a sample comprises bringing the sample into contact with a reaction mixture comprised of a plurality of oligonucleotide primers, at least one oligonucleotide probe, and a plurality of nucleic acid enzymes, such that an amplification reaction occurs to produce a plurality of amplicons when the target nucleic acid is present, wherein i) the plurality of oligonucleotide primers is designed for amplifying a region of the target nucleic acid, whereby the amplicons are produced; ii) the probe contains a scissile linkage and hybridizes to an amplicon iii) the plurality of nucleic acid enzymes comprises at least a first enzyme having template-dependent nucleic acid polymerase activity and a second enzyme capable of selectively cleaving the scissile linkage, and then (B) detecting whether the amplicons are present in the reaction mixture. In one aspect, a scissile linkage is capable of being cleaved or disrupted without cleaving or disrupting any nucleic acid sequence of the molecule itself, or of the target nucleic acid sequence. A scissile linkage can comprise any connecting chemical structure which joins two nucleic acid sequences and which is capable of being selectively cleaved without cleavage of the nucleic acid sequences to which it is joined. In one example, a scissile linkage is RNA when the flanking sequences are DNA. For this example, RNase H is a preferred second enzyme.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference in their entirety.

### EXAMPLES

### Example 1

This example demonstrates the detection of HPV-16 amplicons produced by LAMP or PCR.

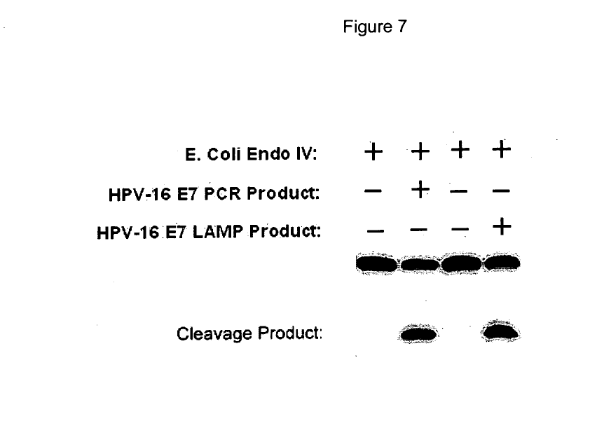

HPV-16 E7 DNA was amplified using standard PCR and LAMP protocols. Afterwards, amplicons were detected using a nucleic acid repair enzyme and an oligonucleotide probe that contained a mismatched or repairable base sequence. As depicted in FIG. 7, experimental samples alternatively contained the nucleic acid repair enzyme E. coli endonuclease IV, HPV-16 PCR amplicons, or HPV-16 LAMP amplicons. Each sample contained 10 units of E. coli endonuclease IV, 10 pmoles of ³²P-labeled HPV-16 E7 probe containing an internal propanyl AP site (C Spacer), and 50 fmoles of HPV-16 E7 amplicons. Samples of 20 μl were incubated at 37° C. for 60 minutes and analyzed by 20% denaturing PAGE.

As shown in FIG. 7, E. coli endonuclease IV cleaves an HPV-16 E7 probe containing a propanyl C Spacer in the presence of a LAMP or PCR product thereby detecting the presence of HPV-16 nucleic acids.

### Example 2

This example demonstrates the detection of HPV-16 amplicons produced by LAMP in the presence of a nucleic acid repair enzyme and an oligonucleotide probe.

The nucleic acid repair enzyme E. coli endonuclease IV and a radiolabeled HPV-16 E7 probe containing an artificial propanyl AP site were included in a LAMP reaction mixture with 100 ng of Caski genomic DNA. The DNA was derived from a human cervical cancer cell line containing about 600 copies of HPV-16 DNA per cell. Each LAMP amplification reaction mixture contained amplification primers for HPV-16 E7, 8 units of Bst DNA polymerase, 10 units of E. coli endonuclease IV, 1.0 pmoles of ³²P-labeled HPV-16 E7 probe containing an internal propanyl AP site, and 100 ng of Caski genomic DNA, where indicated. Samples of 20 μl were incubated at 60° C. for 30 minutes and analyzed by 20% denaturing PAGE.

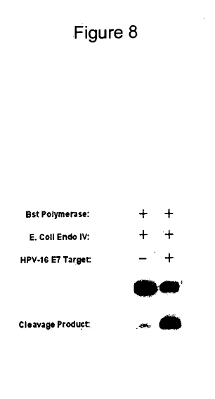

The autoradiograph provided in FIG. 8 shows the cleavage of the oligonucleotide probe which occurred during the amplification reaction.

Likewise, HPV-16 E7 DNA was detected in 10 ng of SiHa genomic DNA, a human cell line that contains one copy of HPV-16 DNA per cell. Each LAMP reaction contained amplification primers for HPV-16 E7, 8 units of Bst DNA polymerase, 10 units of PA-endonuclease IV, 10 pmoles of ³²P-labeled HPV-16 E7 probe containing an internal propanyl AP site, and varying amounts of target SiHa genomic DNA or salmon testes DNA to act as a negative control. Samples of 20 μl were incubated at 60° C. for 30 minutes and analyzed by 20% denaturing PAGE.

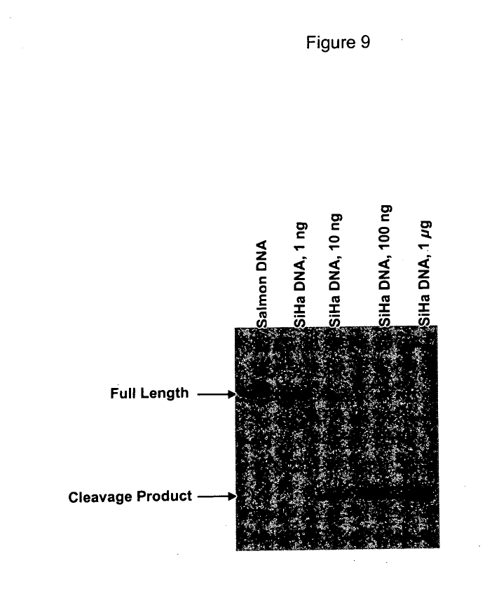

FIG. 9 shows that HPV-16 E7 DNA was detected in as little as 10 ng of SiHa genomic DNA.

### Example 3

This example demonstrates real-time detection of HPV-16 amplicons produced by a LAMP reaction using a nucleic acid repair enzyme and an oligonucleotide probe.

Oligonucleotide probes for HPV-16 E7 containing an internal propanyl C3 spacer were designed. The oligonucleotide probes were labeled with the fluorophore FAM and the quencher BHQ-1 on the 5′ and 3′ ends of the probe, respectively. The probes are depicted in Table 2.

TABLE 2
HPV-16 E7 Oligonucleotide Probes

| | | | 3′ |
|---|---|---|---|
| Probe | 5′ Donor | Sequence | Quencher |
| HPV-16 | FAM | TGTGCCCATTAA-(SpacerC3)- | BHQ1 |
| E7 Probe | | AGGTCTTCCAA | |
| Negative | FAM | TGTGCCCATTAA-C- | BHQ1 |
| Control | | AGGTCTTCCAA | |
| Probe | | | |

The nucleic acid repair enzymes E. coli endonuclease IV or T. maritima endonuclease IV and either the HPV-16 E7 FRET probe or the negative control FRET probe were incorporated directly in a series of LAMP amplification reactions containing 1 μg of SiHa genomic DNA. This DNA was derived from a cervical cancer cell line with about 1 copy of HPV-16 DNA per cell. Each LAMP amplification reaction mixture contained primers for amplification of HPV-16 E7 and 8 units of Bst DNA polymerase in a 25 μl reaction volume. To 15 μl of the LAMP reaction, 10 units of E. coli endonuclease IV, and 20 pmoles of HPV-16 E7 FRET probe, containing an internal propanyl AP site, were added in a volume of 80 μl in 96-well plates. Control reactions contained 0.25 pmol of an oligonucleotide containing the sequence complementary to the E7 FRET probe. Negative control LAMP reactions contained primers for amplification of HPV-16 E7 by LAMP and 8 units of Bst polymerase without genomic DNA. Fluorescence was measured as a function of time throughout the amplification reaction and also at the completion of the LAMP reaction.

Upon completion, the specific increase in fluorescence due to cleavage of the HPV-16 E7 probe was calculated by the following equation:

$$FRET Fluorescence Ratio = \frac{\begin{matrix}Fl ( C 3 Probe with Endo IV - \\ Fl ( C 3 Probe without Endo IV )\end{matrix}}{\begin{matrix}Fl ( Neg . Probe with Endo IV - \\ Fl ( Neg . Probe without Endo IV )\end{matrix}}$$

A FRET ratio greater than 5.0 indicated the presence of HPV-16 E7 DNA. With E. coli endonuclease IV and T. maritima endonuclease IV, the FRET ratio was 28.18 and 5.4, respectively.

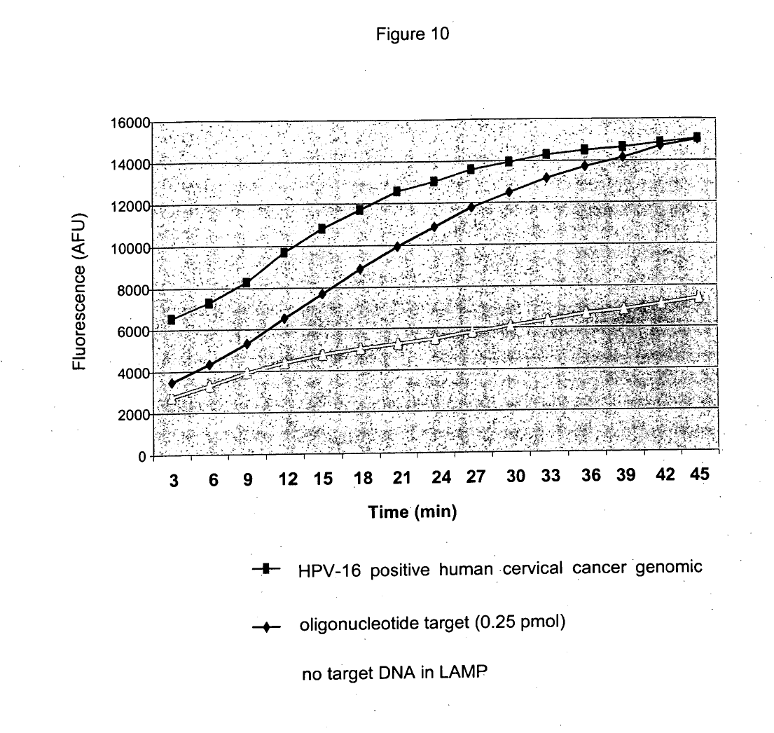

FIGS. 10 and 11 depict the real-time change in fluorescence during a LAMP amplification reaction. Both figures demonstrate that an isothermic, non-specific LAMP amplification reaction can be combined with nucleic acid repair enzymes and labeled oligonucleotide probes to provide rapid, sensitive, and specific detection of target nucleic acids.

While the invention is described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. A method of analyzing for the presence of a target nucleic acid in a sample, said method comprising
(A) bringing said sample into contact with a reaction mixture comprised of a plurality of oligonucleotide primers, at least one oligonucleotide probe, and a plurality of nucleic acid enzymes, such that an amplification reaction occurs to produce a plurality of amplicons when said target nucleic acid is present, wherein
i) said plurality of oligonucleotide primers is designed for amplifying a region of the target nucleic acid, whereby said amplicons are produced;
ii) said probe undergoes hybridization to an amplicon of said plurality of amplicons and contains a mismatched or repairable base sequence such that, in said hybridization, a mismatch occurs at the site of said mismatched or repairable base sequence; and
iii) said plurality of nucleic acid enzymes comprises at least a first enzyme having template-dependent nucleic acid polymerase activity and a second enzyme having nucleic acid repair enzyme activity, and then
(B) detecting whether said amplicons are present in said reaction mixture.

2. The method of claim 1, wherein said amplification reaction is selected from the group consisting of a polymerase chain reaction, a ligase chain reaction, a loop-mediated isothermal amplification, a nucleic acid sequence based amplification, a self-sustained sequence replication, a strand displacement amplification, and a transcription mediated amplification.

3. The method of claim 1, wherein said second enzyme is selected from the group consisting of a mismatch repair enzyme, a glycosylase, an apurinic-apyrimidinic (AP) endonuclease and an AP lyase.

4. A reaction mixture for detecting a target nucleic acid in a sample, said reaction mixture comprising a plurality of oligonucleotide primers, one or more oligonucleotide probes, and a plurality of nucleic acid enzymes, wherein
i) said plurality of oligonucleotide primers are operably designed for amplifying a region of said target nucleic acid;
ii) said one or more oligonucleotide probes hybridizes to an amplicon of said target nucleic acid and contains a mismatched or repairable base sequence such that, in the hybridization, a mismatch occurs at the site of said mismatched or repairable base sequence; and
iii) said plurality of nucleic acid enzymes comprises at least a first enzyme having template-dependent nucleic acid polymerase activity and a second enzyme having nucleic acid repair enzyme activity.

5. The reaction mixture of claim 4, wherein each enzyme of the plurality of enzymes are independently mesophilic or thermophilic.

6. The reaction mixture of claim 4, wherein said second enzyme is selected from the group consisting of a mismatch repair enzyme, a glycosylase, an apurinic-apyrimidinic (AP) endonuclease and an AP lyase.

7. The reaction mixture of claim 6, wherein said glycosylase is selected from the group consisting of Uracil DNA glycosylase, E. coli MutY glycosylase, M. thermoautotrophicum TDG and P. aerophilum MIG.

8. The reaction mixture of claim 6, wherein said AP endonuclease is selected from the group consisting of E. coli endonuclease IV, T. maritima endonuclease IV, P. aerophilum endonuclease IV and M. thermoautotrophicum endonuclease IV.

9. The reaction mixture of claim 6, wherein said plurality of nucleic acid enzymes comprises a third enzyme selected from the group consisting of a mismatch repair enzyme, a glycosylase, an apurinic-apyrimidinic (AP) endonuclease and an AP lyase.

10. The reaction mixture of claim 4, wherein said labeled oligonucleotide probe is detectable by fluorescence.

11. The reaction mixture of claim 4, wherein said oligonucleotide probe comprises a first and second label.

12. The reaction mixture of claim 11, wherein said first and second labels are interactive signal generating labels effectively positioned on the one or more oligonucleotide probes to quench the generation of detectable signal.

13. The reaction mixture of claim 12, wherein said first label is a fluorophore and said second label is a quenching agent.

14. The reaction mixture of claim 10, wherein said first label is at the 5′ terminus of said oligonucleotide probe and said second label is at the 3′ terminus of said oligonucleotide probe.

15. The reaction mixture of claim 10, wherein said first and second labels are located internally within said oligonucleotide probe on opposite sides of said mismatched or repairable base sequence.

16. A kit for detecting a target nucleic acid in a sample, said kit comprising a plurality of oligonucleotide primers, an oligonucleotide probe, and a plurality of nucleic acid enzymes, wherein
i) said plurality of oligonucleotide primers are operably designed for amplifying a region of said target nucleic acid;
ii) said one or more oligonucleotide probes hybridize to an amplicon of said target nucleic acid and contains a mismatched or repairable base sequence such that, in the hybridization, a mismatch occurs at the site of said mismatched or repairable base sequence; and
iii) said plurality of nucleic acid enzymes comprises at least a first enzyme having template-dependent nucleic acid polymerase activity and a second enzyme having nucleic acid repair enzyme activity.

17. The kit of claim 16, wherein each enzyme of said plurality of enzymes are independently mesophilic or thermophilic.

18. The kit of claim 16, wherein said second enzyme is selected from the group consisting of a mismatch repair enzyme, a glycosylase, an apurinic-apyrimidinic (AP) endonuclease and an AP lyase.

19. The kit of claim 18, wherein said glycosylase is selected from the group consisting of Uracil DNA glycosylase, E. coli MutY glycosylase, M. thermoautotrophicum TDG and P. aerophilum MIG.

20. The kit of claim 18, wherein said plurality of nucleic acid enzymes comprises a third enzyme selected from the group consisting of a mismatch repair enzyme, a glycosylase, an apurinic-apyrimidinic (AP) endonuclease and an AP lyase.

21. The kit of claim 18, wherein said AP endonuclease is selected from the group consisting of E. coli endonuclease IV, T. maritima endonuclease IV, P. aerophilum endonuclease IV and M. thermoautotrophicum endonuclease IV.

22. The kit of claim 16, wherein said plurality of nucleic acid enzymes comprises a third enzyme selected from the group consisting of a mismatch repair enzyme, a glycosylase, an apurinic-apyrimidinic (AP) endonuclease and an AP lyase.

23. The kit of claim 16, wherein said labeled oligonucleotide probe is detectable by fluorescence.

24. The kit of claim 16, wherein said oligonucleotide probe comprises a first and second label.

25. The kit of claim 24, wherein said first and second labels are interactive signal generating labels effectively positioned on the one or more oligonucleotide probes to quench the generation of detectable signal.

26. The kit of claim 25, wherein said first label is a fluorophore and said second label is a quenching agent.

27. The kit of claim 24, wherein said first label is at the 5′ terminus of said oligonucleotide probe and said second label is at the 3′ terminus of said oligonucleotide probe.

28. The kit of claim 24, wherein said first and second labels are located internally within said oligonucleotide probe on opposite sides of said mismatched or repairable base sequence.


SEQUENCE LISTING (2 sequences)

SEQ ID NO: 1 - DNA, 26 bp, Artificial Sequence
  Description of Artificial Sequence Synthetic probe
tgtgcccattaacccaggtcttccaa

SEQ ID NO: 2 - DNA, 24 bp, Artificial Sequence
  Description of Artificial Sequence Synthetic probe
tgtgcccattaacaggtcttccaa